(19) United States
(12) Patent Application Publication
(10) Pub. No.: US 2022/0042108 A1
(43) Pub. Date: Feb. 10, 2022

(54) SYSTEMS AND METHODS OF ASSESSING BREAST CANCER

(71) Applicant: Agendia NV, Amsterdam (NL)

(72) Inventors: Bastiaan Van Der Baan, Irvine, CA (US); Annuska Maria Glas, Irvine, CA (US)

(21) Appl. No.: 17/395,033

(22) Filed: Aug. 5, 2021

Related U.S. Application Data
(60) Provisional application No. 63/062,126, filed on Aug. 6, 2020.

(51) Int. Cl. C12Q 1/6886

(52) U.S. Cl. CPC C12Q 1/6886; C12Q 2600/156

(57) ABSTRACT

This disclosure relates to systems and methods for providing individualized assessments of breast cancer. The assessments are provided in the format of index scores that are prognostic to disease severity and progression. Methods involve measuring gene expression from a patient sample to create a gene expression signature. The gene expression signature is compared with expression signatures from tumors that are associated with known outcomes. The comparison is used to generate an index score that assigns a risk status to the patient. The index score may indicate, for example, risk of distant metastasis or benefit of chemotherapy.

### TECHNICAL FIELD

This invention relates to oncology. In particular, this invention provides systems and methods for assessing breast cancer prognosis.

### BACKGROUND

Breast cancer is clinically heterogeneous. Patients presenting identical symptoms may differ widely with respect to their treatment outcomes. This clinical heterogeneity may be due to complex genetic differences among individuals. For example, genetic differences may influence the expression of genes involved in key aspects of tumor growth, thereby giving rise to various treatment responses.

Clinical and histopathologic features such as age, tumor size, histologic tumor type, pathologic grade, estrogen receptor (ER) status, and lymph node status, are frequently used to assess risk of risk of recurrence and residual disease. Recently, clinical tools available for assessing cancer have expanded to include analyses of genetic features. For example, DNA microarrays have been used to obtain genome-wide views of human tumor gene expression, and these studies have identified cancer biomarkers with diagnostic, prognostic, and predictive potential in a wide variety of solid tumors, including breast cancer.

Accurate assessment of individualized risk remains an important goal. Despite the numerous tools available, making an accurate assessment remains difficult on account of the complexity of the disease. For example, different markers of clinical pathology may vary widely in significance depending on a tumor subtype. The physician is left to search the literature to interpret the results of individual patient assessments.

### SUMMARY

This disclosure provides individualized assessments of breast cancer patients. The assessments are provided in the format of index scores that give important, and easy to understand, information about patient tumors. Preferably, the index score is based on measurements of cell-free nucleic acids taken from a patient fluid sample (e.g., blood or plasma). The measurements are used to create gene expression signatures that are correlated with expression signatures from tumors that have known treatment outcomes to create an index score. Because the index score is created based on correlations with known outcomes, the index score can assign a risk status to the patient that is predictive of disease severity or progression. The index score may indicate, for example, risk of cancer metastasis or predicted benefit of an aggressive treatment such as chemotherapy.

Preferably, the index score is provided as an easy-to-understand score. For example, the index score may be provided on an easily interpretable scale due to the index score being a numerical value between −1.0 and 1.0. The easily interpretable scale may make talking to patients about their test results easy and efficient. It may also help the physician make treatment decisions more quickly by reducing the amount of time required to interrupt patient results. Methods of the invention may further include combining expression signatures with other clinical factors to give a single risk appraisal.

Methods of the invention create index scores useful for the clinical management of breast cancer. The index scores may predict, for example, a risk of disease recurrence or metastasis, and as such, the index scores may be used to select an optimal course of treatment. For example, the index scores may be used to identify patients that are at a high risk for recurrence and thus good candidates for aggressive treatment options such as chemotherapy. Accordingly, index scores may be useful for classifying a patient and selecting an appropriate treatment.

In one aspect, this disclosure provides a method for assessing disease. The method includes identifying RNA expression levels present in a tissue or body fluid sample obtained from a cancer patient. Preferably, RNA expression levels are identified from a body fluid sample, which offers an avenue for making non-invasive disease management. The RNA expression levels are compared, and correlated, with expression levels that are expected in one or more stages of cancer to create an index score. The index score provides an individualized assessment of risk, which is useful for assessing the disease severity and progression. The index score may indicate that the patient is at a low risk of having a metastatic event or recurrence of disease. For example, the index score may indicate that a patient is at a low risk of distant metastasis within 5 or 10 years.

The RNA may be obtained from patient tissue or body fluids. Preferably, the RNA is obtained from a body fluid sample to avoid a painful biopsy. Moreover, by obtaining RNA from a body fluid sample, assessments of risk for recurrence or metastasis may be made at any point during treatment. For example, assessments may be made before and/or after a tumor is removed. This allows for longitudinal disease management. The body fluid sample may comprise one of blood, saliva, sputum, urine, semen, transvaginal fluid, cerebrospinal fluid, or sweat.

Preferably, the body fluid sample comprises a blood sample. One insight of the invention is that RNA is surprisingly stable in blood when encapsulated inside extracellular vesicles where they and are protected from degradation. As such, methods of the invention may include isolating an extracellular vesicle from a blood sample and identifying RNA expression levels for the contents of the vesicle.

Methods of the invention rely on identifying RNA expression levels to create index scores that are prognostic towards disease severity and progression. Identifying RNA expression levels may include making quantitative measurements of different species of RNA. The RNA preferably comprises messenger RNA (mRNA), and more preferably, the mRNA includes one or more gene transcripts associated with cancer, such as breast cancer. For example, the mRNA may comprise transcripts of genes associated with those that are probed for in diagnostic breast cancer assays, such as the cancer assays sold under trade names MammaPrint and/or BluePrint by Agendia, Inc. Accordingly, identified levels of RNA from the patient may be diagnostic with respect to breast cancer.

Measuring amounts of RNA molecules may involve interrogating a sample with probes specific for transcripts derived from a panel of genes, e.g., oncogenes, and measuring expression levels for positive probe responses. Advantageously, this allows a researcher or clinician to focus their analysis on RNA with positive clinical value and avoids wasting time and recourses processing material that is of no or little value. The panel of genes may include genes associated with breast cancer or involved in hormone receptor regulation.

A preferred method of the invention comprises creating a cDNA copy of each molecule of RNA and then sequencing the cDNA copies to generate a plurality of sequencing reads. Sequencing may be accomplished using any standard sequencing technology, but preferably involves next generation sequencing technologies. The sequencing reads may be analyzed to determine expression levels of distinct species of RNA. The expression levels are compared and correlated with expression levels expected in one or more stages of cancer to create an index score that is predictive of disease severity, which is then used to provide a cancer prognosis.

Methods of the invention may include analyzing an image from a stained tissue sample to support of confirm a prognosis. For example, the image may be an image of a tumor sample from the patient and stained with, for example, a H&E stain, Pap stain, an immunohistochemical stain, or any other suitable staining/labelling media. The staining may reveal specific molecular markers that are indicative of disease stage and progression. For example, immunohistochemistry staining may be used to reveal intracellular proteins characteristic of a tumor subtype. Accordingly, methods of the invention include obtaining an image of a stained tissue sample from a patient; and analyzing the image to detect one or more features indicative of disease severity to support or confirm a prognosis.

In some instances, methods of the invention may implement analysis systems, such as machine learning systems. Methods may include providing expression data from a patient as an input to an analysis system trained on training data comprising one or more sets of training expression level measurements associated with known patient outcomes. Preferably, the analysis system comprises a computer system with a machine learning algorithm. The analysis system may be a machine learning system. Using the power of machine learning, the methods and systems of the invention can leverage vast amounts of old and/or new data to provide a more informed and accurate individual assessment of risk.

Furthermore, other data, such as image data from the patient may be provided as part of the inputs to the analysis system. The methods and systems of the disclosure can analyze this disparate data, such as expression levels of nucleic acids and image data, in combination to provide correlative prognoses. The methods and systems of the disclosure may include an analysis system hosting a trained machine learning algorithm. Image data provided as an input may be an image of a stained, FFPE slide from a tumor from the patient.

### BRIEF DESCRIPTION OF THE DRAWINGS

FIG. 1 diagrams a method for assessing cancer prognosis.

FIG. 2 shows a body fluid sample.

FIG. 3 diagrams a method of sample prep.

FIG. 4 shows an analysis system.

### DETAILED DESCRIPTION

This disclosure relates to systems and methods for providing individualized assessments of breast cancer. The assessments are provided in the format of index scores that are prognostic to disease severity and progression. In particular, systems and methods involve measuring gene expression from a patient sample to create a gene expression signature. The gene expression signature is compared with expression signatures from tumors that are associated with known outcomes. The comparison is used to generate an index score that assigns a risk status to the patient. The index score may indicate, for example, risk of distant metastasis or benefit of chemotherapy. The index score will allow a physician to quickly make a treatment decision with confidence. Methods of the invention may further include combining expression signatures with other clinical factors to give a single risk appraisal.

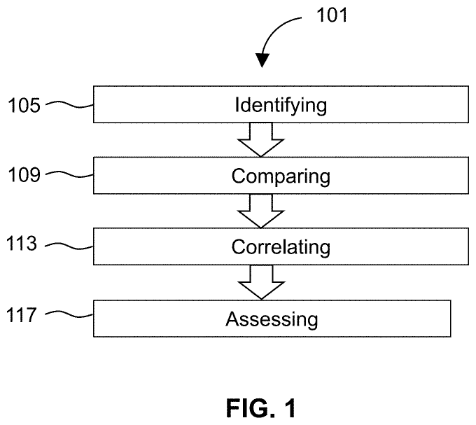

FIG. 1 diagrams a method 101 for assessing cancer prognosis. The method includes identifying 105 RNA expression levels present in a tissue or body fluid sample obtained from a cancer patient and comparing 109 the expression levels to expression levels expected in one or more stages of cancer. The method further includes correlating 113 the identified expression levels with said expression levels expected in the one or more stages of cancer to generate an index score and assessing 117 cancer prognoses based on the index score.

RNA expression levels may be identified 105 from a tissue sample or a body fluid sample. Preferably, the RNA expression levels are identified 105 from a body fluid sample. Identifying 105 RNA expression levels from a body fluid sample, as opposed to a solid tissue sample, provides an avenue for longitudinal disease monitoring. For example, RNA may be identified 105 from multiple body fluid samples taken from the same patient overtime to monitor changes in disease severity or progression. Moreover, because a tumor biopsy is not required, disease assessments may be made before the patient exhibits any signs or symptoms of cancer.

The body fluid sample may comprise one of blood, saliva, sputum, urine, semen, transvaginal fluid, cerebrospinal fluid, sweat, stool, a cell or a tissue. Preferably, the sample comprises blood, as it is an insight of the invention that molecules of RNA are surprisingly stable in blood when encapsulated inside extracellular vesicles where they are protected from degradation.

Preferably, the body fluid sample is taken from a patient that is suspected of having a disease, such as cancer. The patient may be suspected of having a cancer on account of various symptoms including the detection of a lump or mass. The cancer may be one of bladder cancer; breast cancer; colorectal cancer; kidney cancer; lung cancer; lymphoma; skin cancer; oral cancer; pancreatic cancer; prostate cancer; thyroid cancer; or uterine cancer. More preferably, the cancer is early stage breast cancer, i.e., cancer that is contained entirely within the breast.

According to aspects of the invention, molecules of RNA may be identified 105, e.g., detected and quantified, by any of a wide variety of methods, including, but not limited to, sequencing (e.g., RNA-seq), hybridization analysis, amplification e.g., via the polymerase chain reaction, for example, by reverse transcription polymerase chain reaction (RT-PCR).

Identifying 105 RNA expression levels may involve targeted enrichment next-generation sequencing technologies, which are useful for measuring RNA expression levels of specific RNA transcripts of interest. Specific transcripts of interest may include, for example, transcripts of genes probed by the diagnostic breast cancer assays sold under the trade names MammaPrint and BluePrint, by Agendia, Inc. For example, as described in Mittempergher, 2019, MammaPrint and BluePrint Molecular Diagnostics Using Targeted RNA Next-Generation Sequencing Technology, The Journal of Molecular Diagnostics, Volume 21, Issue 5, 808-823, which is incorporated by reference.

Identifying 105 RNA expression levels may involve isolating RNA from the patient sample. The RNA may be uniquely barcoded RNA and converted into complementary DNA (cDNA). Specific cDNA molecules may be probed for using biotinylated capture RNA baits. The captured cDNA molecules may be analyzed by sequencing to produce a plurality of sequence reads. The plurality of sequence reads may be de-duplicated based on their unique barcodes and mapped to a reference genome to identify their genetic loci origin. Unique sequence reads that map to each locus of the reference genome are counted to identify 105 RNA expression levels.

Preferably, the RNA expression levels comprise expression levels of gene transcripts that are differentially expressed in caner, and more preferably, the RNA expression levels comprise expression levels for the genes evaluated by MammaPrint and/or BluePrint, for example, as described in U.S. Pat. No. 10,072,301 and WO2002/103320, which are incorporated herein by reference.

After identifying 105 RNA expression levels, the expression levels are compared 109 to expression levels expected in one or more stages of cancer.

Comparing 109 may be performed with an unsupervised, hierarchical clustering algorithm, such as a K-means clustering algorithm. A clustering algorithm is an algorithm that clusters or groups a set of objects in such a way that the objects in the same group (called a cluster) are more like each other than to those in other groups (clusters). The clustering algorithm may cluster RNA expression levels from the patient sample with the RNA expression levels expected in one or more stages of cancer. The RNA expression levels expected in one or more stages of cancer may come from one or more tumor samples associated with known outcomes. The RNA expression levels may be clustered based on their similarities of expression. Preferably, the clustering algorithm clusters the RNA expression levels into distinct groups associated with the known outcomes, for example, as discussed in van′t Veer, 2002, Gene Expression Profiling Predicts Clinical Outcome of Breast Cancer, Nature, Vol 415, pages 530-535, which is incorporated by reference. The groups may reflect a continuum of outcomes that are indicative of prognoses. For example, one group may be reflective of a poor prognosis (e.g., metastasis within 5-years of treatment), one group may be reflective of a moderate prognosis, one group may be reflective of a good prognosis, etc.

The RNA expression levels from the patient may be correlated 113 with the grouped expression levels from the tumor samples to create an index score. Preferably, the correlating 113 step is based on the comparing 109 step. Correlating 113 may involve, for example, determining a Pearson correlation between the RNA expression levels of genes in the patient sample and the expression levels of genes from the tumor samples for each of the one or more known outcomes. The Pearson correlation between the expression levels of the genes in the patient sample and the expression levels in a sample is used to create the index.

The index score may vary between +1, indicating a prefect similarity, and −1, indicating a reverse similarity. The index score may be displayed or outputted to a user interface device, a computer readable storage medium, or a local or remote computer system.

Preferably, the comparing 109 and correlating steps 113 are performed exclusively on RNA that is expressed at levels substantially above a pre-determined threshold that is identified as background noise. For example, on RNA that is expressed at least 1-fold, 2-fold, 3-fold, 4-fold, 5-fold, 6-fold or 7-fold above the level of expression that is identified as background noise. Identifying the threshold associated with background noise may be done according to methods routinely used in the art. Analyzing RNA that is expressed at levels substantially above background noise may produce more stable gene signatures, and thus, provide more accurate prognostic information.

Methods of the invention may use index scores to predict how well a given patient will respond to certain treatments. Because methods of the invention are useful for predicting how well a patient will respond to certain treatments, an effective treatment may be recommended to the patient, and clinicians can avoid spending the time and money on treatment protocols that will not help the patient. Recommending a treatment may involve selecting one or more drugs likely to be effective for treating the patient.

Because an effective treatment is given to the patient rapidly, the patient with a tumor or an early stage cancer will have a good chance of remission and recovery. Selecting a course of treatment may further involve identifying a drug that a patient is likely to respond to by, for example, determining or predicting a response of the patient to the treatment. Accordingly, in some embodiments methods and systems of the invention involve identifying and grouping cell-free nucleic acids to determine that a patient cancer will be effectively treated or cured by administering one or more specific drugs. In some embodiments, selecting a course of treatment involves determining that a patient needs a tumor resection.

Preferably, the index score is provided as an easy-to-understand score. For example, the index score may be provided on an easily interpretable scale due to the index score being a numerical value between, for example, −1.0 and 1.0, −10.0-10.0, or any other numerical range. Alternatively, the index score may comprise a color, or a symbol, or the like. The easily interpretable scale may make talking to patients about their test results easy and efficient. It may also help the physician make treatment decisions more quickly as the physician does not need to waste time interpreting the score.

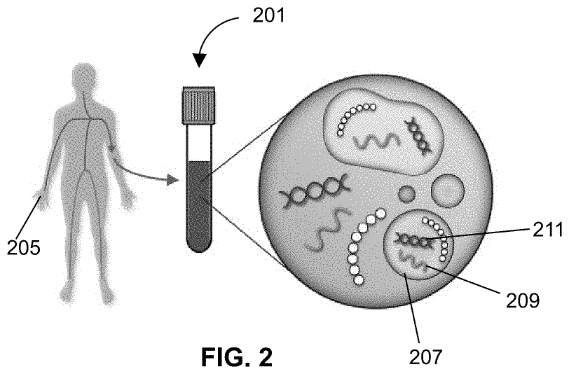

FIG. 2 shows a body fluid sample 201. The body fluid sample 201 comprises blood 203 and is preferably taken from a patient 205 by blood draw. The blood 203 may include extracellular vesicles 207. Extracellular vesicles 207 are small plasma membrane-encapsulated particles, which comprise exosomes and microvesicles, that are released by all cells and that can enter the bloodstream. Extracellular vesicles 207 are ubiquitous in body fluids including blood plasma, cerebral spinal fluid, aqueous humor, amniotic fluid, saliva, synovial fluid, adipose tissue, and urine. Both blood plasma and cerebral spinal fluid extracellular vesicles including exosomes are a useful source of cell-free nucleic acids for assessing disease.

Extracellular vesicles 207 contain proteins (tumor antigens, immunosuppressive, and/or angiogenic molecules) and cell-free nucleic acids, including cell free RNA 209 and cell free DNA 211 specific to cancer cells. Thus, their cargo may be analyzed to determine their cell of origin by, for example, by segregating the extracellular vesicles 207 and sequencing the nucleic acids contained therein or performing an immunochemistry staining for cell-type specific proteins. In some cases, the extracellular vesicles 207 may be segregated by immunostaining the extracellular vesicles 207 for a protein that is over or under expressed in cancer, and subsequently sorting the stained extracellular vesicles 207 by FACS.

Methods of the invention may include determining an extracellular vesicle's origin (e.g., determining that the vesicle was released from a tumor cell) based on the content of the extracellular vesicle before identifying at least two of the cell-free nucleic acids contained therein, as described below. By determining the extracellular vesicle's origin prior to identifying the cell-free nucleic acids, a researcher or clinician, may focus their analyses specifically on nucleic acids associated with tumor cells. Accordingly, methods of the invention allow for the analysis of cargo of extracellular, after those extracellular vesicles have been isolated form a blood or plasma sample form the patient, to thereby assess the tumor.

The extracellular vesicles may be isolated from blood collected by blood draw or by fine needle aspiration. Isolating the extracellular vesicles from the body fluid sample may involve a differential ultracentrifugation (low-speed centrifugation to remove cells and debris, high-speed ultracentrifugation to pellet exosomes). For example, to isolate extracellular vesicles from blood the sample, may be centrifuged at low speeds allowing for the removal of cells and debris by, for example, pipetting or dumping out supernatant. The sample may then be centrifuged at high speeds, for example, at 100,000×g for 70 min, to pellet the extracellular vesicles allowing the extracellular vesicles to be separated from remaining material. Easy-to-use precipitation solutions, such as the precipitation solution sold under the trade name ExoQuick by System Biosciences, may be used to precipitate the vesicles in liquid. Once the vesicles are isolated, the vesicles may be lysed in lysis buffer to release the cell-free nucleic acids. For example, as described Garcia, 2019, Isolation and Analysis of Plasma-Derived Exosomes in Patients With Glioma, Front Oncol, 9: 651, incorporated by reference.

The RNA collected from the extracellular vesicles may be referred to as cell-free (cfRNA), which may include messenger RNA (mRNA), microRNA (miRNA), long non-coding RNA (lncRNA), and circular RNA (circRNA). The cfRNA may or may not be fragmented to a desired size. Fragmenting may be performed using sonication methods or by enzyme treatment. Preferably, the isolated cfRNA comprises a 260/280 and 260/230 absorbance ratio values of close to 2.0. Once the cfRNA are isolated, a cfRNA sample prep procedure may be performed to identify the cfRNA.

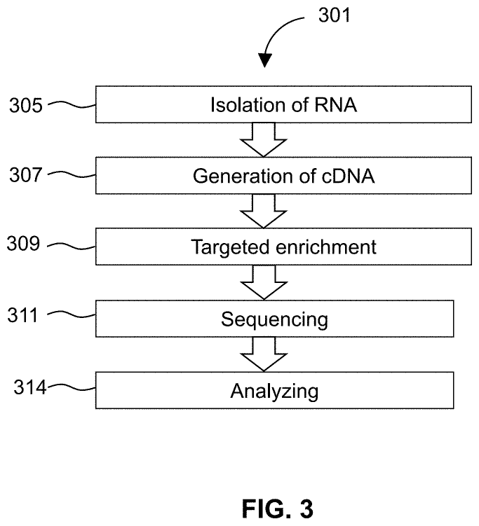

FIG. 3 diagrams a method 301 of sample prep according to aspects of the invention. The method 301 includes isolating 305 RNA. Preferably, the RNA comprises cfRNA. The cfRNA is preferably isolated from extracellular vesicles collected in a blood sample. In some embodiments, RNA isolation 305 is performed with an RNA isolation kit sold, such as the RNA isolation kit sold under the trade name RNeasy by Qiagen (Valencia, Calif.), and in accordance with the manufacturer's instructions. Isolated cfRNA preferably has a 260/280 and 260/230 absorbance ratio values close to 2.0. To determine the quality of the RNA, a nucleic acid analysis system, such as the Agilent 2100 Bioanalyzer instrument, may be used. In some embodiments, the cfRNA may be chemically fragmented. Preferably, the fragments comprise 200 base pairs.

Following isolation 305, the RNA is converted to cDNA. The generation of cDNA 307 can be done by a variety of methods, but, preferably, the cDNA is generated using reverse transcriptase, which can use the information in a molecule of RNA to generate a molecule of cDNA. Reverse transcriptase is a RNA-dependent DNA polymerase. Like all DNA polymerases it cannot initiate synthesis de novo but depends on the presence of a primer. Since many RNAs have a poly-A tail at the 3′ end, oligo-dT is frequently used to prime DNA synthesis. It is also possible, and frequently essential, to generate cDNAs by using either random primers or primers designed to amplify a specific RNA.

Once a first strand of cDNA has been created, it is generally necessary to produce a second strand of DNA. A person of skill in the art will recognize that there are many methods for producing the second strand, but a convenient mechanism involves exposure of the DNA/RNA hybrid to a combination of RNAase-H and DNA polymerase. RNAase-H has the ability to cause single-stranded nicks in the RNA, and DNA polymerase can then use these single-stranded nicks to initiate “second strand” DNA synthesis. This two-step procedure has been optimized to maximize fidelity and length of cDNAs. In preferred embodiments, adapters are ligated onto the ends of the cDNA. The cDNA may be adenylated at the 3′ end prior to adapter ligation. Preferably, the adapters comprise sequencing platform specific primers, such as the Illumina P5/P7 (flow cell binding primers). The adapters may also comprise PCR primer biding sites for amplifying the cDNA library.

In some embodiments, the adapters may further include barcode sequences. The barcode sequences may be used to give each molecule of cDNA a unique tag, e.g., a unique molecular identifier. Unique molecular identifiers or molecular barcodes are short DNA molecules which may be ligated onto DNA fragments, e.g., cDNA fragments. The random sequence composition of the unique molecular identifiers assures that every fragment-unique molecular identifier combination is unique in the library. Thus, after PCR amplification, it is possible to distinguish multiple copies of a fragment caused by PCR clones versus real biological duplications. By using unique molecular identifiers, PCR clones can be found by searching for non-unique fragment-UMI combinations, which can only be explained by PCR clones. Following adapter ligation, the cDNA may be amplified by PCR.

Preferably, biotinylated capture baits or probes are used for the targeted enrichment 309 of specific cDNA molecules of interest. The biotinylated capture probes may comprise RNA, DNA, or a hybrid of RNA and DNA nucleotides. Preferably, the capture probes comprise biotinylated RNA, which may provide better signal to noise ratios. The biotinylated RNA capture probes may be added to the cDNA library and incubated for a time period and at a temperature sufficient for the biotinylated RNA capture probes to hybridize to their target molecules of cDNA based on Watson-Crick base pairing. For example, the mixture containing cDNA and probes may be incubated at 65 degrees Celsius for 24 hours. After hybridization, the biotinylated RNA capture probes that are hybridized with the target cDNA molecules may be captured and segregated using streptavidin or an antibody. In preferred embodiments, the target cDNA molecules are amplified by PCR.

The library may then be sequenced 311. An example of a sequencing technology that can be used is Illumina sequencing. Illumina sequencing is based on the amplification of DNA on a solid surface using fold-back PCR and anchored primers. Genomic DNA is fragmented and attached to the surface of flow cell channels. Four fluorophore-labeled, reversibly terminating nucleotides are used to perform sequential sequencing. After nucleotide incorporation, a laser is used to excite the fluorophores, and an image is captured and the identity of the first base is recorded. Sequencing according to this technology is described in U.S. Pub. 2011/0009278, U.S. Pub. 2007/0114362, U.S. Pub. 2006/0024681, U.S. Pub. 2006/0292611, U.S. Pat. No. 7,960,120, U.S. Pat. Nos. 7,835,871, 7,232,656, 7,598,035, 6,306,597, 6,210,891, 6,828,100, 6,833,246, and 6,911,345, each incorporated by reference. In preferred embodiments, an Illumina Mi-Seq sequencer is used. The Ilumina Mi-Seq sequencer is used to generate a plurality of sequence reads that may be uploaded to a web portal for analysis by, for example, the Agendia Data Analaysis Pipeline Tool (ADAPT).

Analyzing 314 the sequence reads may be performed using known software and following a multistep procedure known in the art. For example, first, the quality of each sequence read, i.e., FASTQ sequence, may be assessed using the software FASTQC. Next, the reads may be trimmed by, for example, Trimmomatic software. The trimmed sequence reads may then be mapped to a human genome using the HISAT2 software. HISAT2 output files in a SAM (sequence alignment/map format), which may be compressed to binary sequence alignment/map files using SAMtools version prior sequence read quantification. Afterward, mapped reads may be counted using the feature Counts software.

It may be helpful to support disease prognoses made from analysis of expression levels with other data types that are indicative of disease state or progression.

One other data type that may be used in methods of the disclosure is imaging data, such as histopathology data, e.g., whole-slide imaging. Image data taken from stained tissue samples has long been used to diagnose breast cancer, including subtypes, stage, and prognoses. By combining image data with expression levels of cell free nucleic acids, a more accurate and complete picture of a patient's breast cancer can be produced.

Image data taken from stained tissues is a valuable tool for the detection and evaluation of abnormal cells such as those found in cancerous tumors. By using specific molecular markers that are characteristic of cellular events, such as, proliferation or cell death (apoptosis), a patient tissue sample can be evaluated to determine disease severity. Accordingly, methods of the invention may include obtaining an image of a stained tissue sample from the patient and analyzing the image to detect one or more features indicative of disease severity to support or confirm an assessment of disease severity or progression. The tissue sample may be obtained by biopsy. The biopsy sample may then be stained with markers that label features of disease. For example, the image may be an image of a tumor sample stained with a H&E stain, Pap stain, or any other suitable staining/labelling media. The image may be a digital scan of a stained tissue sample.

The tissue sample may comprise a tissue slice harvested from a patient. The tissue slice may contain information regarding the pathological status of the tissue. Alternatively, the tissue may comprise cells collected by, for example a biopsy, and deposited onto a slide. The cells may include any human cell type, such as, for example, lymphocytes, erythrocytes, macrophages, T-cells, skin cells, fibroblasts, epithelial cells, blood cells, etc. The tissue is imaged with, for example, a high-powered microscope to create image data.

In the methods and systems of the disclosure several features from image data may be assessed, for example, the spatial arrangements and architecture of different types of tissue elements. This can include, by way of example, global features of the epithelial and stromal regions, diversity of nuclear shape, orientation, texture, and architecture, glandular architecture, tumor infiltrating lymphocytes, lymphocyte proximity to cancer cells, the ratio of intratumoural lymphocytes to cancer cells, the tumor stroma, etc.

Methods of the disclosure may use machine learning in conjunction with RNA expression levels to assess breast cancer. This includes, not only providing a diagnosis or prognosis based on known expression transcript signatures, but also creating novel correlations between expression transcripts and other data. Machine learning is branch of computer science in which machine-based approaches are used to make predictions. Bera et al., 2019, Nat Rev Clin Oncol., 16(11):703-715, incorporated by reference. Machine learning-based approaches involve a system learning from data fed into it and use this data to make and/or refine predictions. Machine learning is distinct from traditional, rule-based or statistics-based program models. Rajkomar et al., 2019, N Engl J Med, 380:1347-58, incorporated by reference. Rule-based program models require software engineers to code explicit rules, relationships, and correlations. For example, in the medical context, a physician may input a patient's symptoms and current medications into a rule-based program. In response, the program will provide a suggested treatment based upon preconfigured rules.

In contrast, and as a generalization, in machine learning a model learns from examples fed into it. Over time, the machine learning model learns from these examples and creates new models and routines based on acquired information. As a result, the machine learning model may create new correlations, relationships, routines or processes never contemplated by a human. A subset of machine learning is deep learning. Deep learning uses artificial neural networks. A deep learning network generally comprises layers of artificial neural networks. These layers may include an input layer, an output layer, and multiple hidden layers. Deep learning has been shown to learn and form relationships that exceed the capabilities of humans.

By combining the ability of machine learning, including deep learning, to develop novel routines, correlations, relationships and processes amongst vast data sets of disease biomarker features and patients' clinical data features, (e.g., expression levels and image data) the methods and systems of the disclosure can provide accurate diagnoses, prognoses, and treatment suggestions tailored to specific patients and patient groups afflicted with diseases, including breast cancer.

In some embodiments, methods of the invention exploit the correlative powers of machine learning to assess risk of disease recurrence or metastasis. For example, methods may include providing identified RNA expression levels as inputs to an analysis system that is trained on training data comprising one or more sets of training expression level measurements associated with known patient outcomes. Preferably, the analysis system comprises a computer system with a machine learning algorithm. The analysis system may be a machine learning system. Any suitable machine learning system may be trained using the training data and used to analyze expression levels input into the system. The analysis system may, for example, analyze expression levels to autonomously predict disease severity or progression based on learned correlations with training expression level measurements and known outcomes. The analysis system may report an index score.

In some embodiments, methods of the invention may further include providing an image of a stained tissue from the patient as part of the inputs to the analysis system, wherein the analysis system analyzes the image in combination with the expression levels to assess disease severity or a response to a treatment. For example, tissue images may be obtained from multiple sources and used to train a machine learning system to monitor and diagnose disease.

Methods of the invention may have applicability to deep learning networks and/or unsupervised learning networks that employ data-driven feature representation. Important clinical features of a disease may be represented at nodes within a hidden layer within such a network. Embodiments, a machine learning system is trained and then used to predict how well a given patient will respond to certain treatments. In certain aspects, the invention provides methods that include providing training data to a machine learning system. Training data includes expression levels associated with known outcomes and multiple sets of tissue images that differ in one or more aspects such as tissue type, staining technique, or image capture process. A machine learning system is then trained to recognize features associated with a disease using the training data. Methods of the invention preferably include correlating a prognosis or diagnosis of a disease from expression levels of nucleic acids derived from a patient and, in some instances, a sample tissue image (such as an image of a section from a tumor) from a patient when the machine learning system detects the features in the sample tissue image.

Methods may include generating a report that identifies indicia of disease, includes the prognosis for the cancer for the patient, include a diagnosis, or gives a prediction of a response to a treatment. A prognosis may include a probability of metastasis or recurrence. Methods of the invention may optionally include processing one or more of the images of the training data prior to providing the training data to the machine learning system, in which the processing, for example, removes noise or performs color normalization.

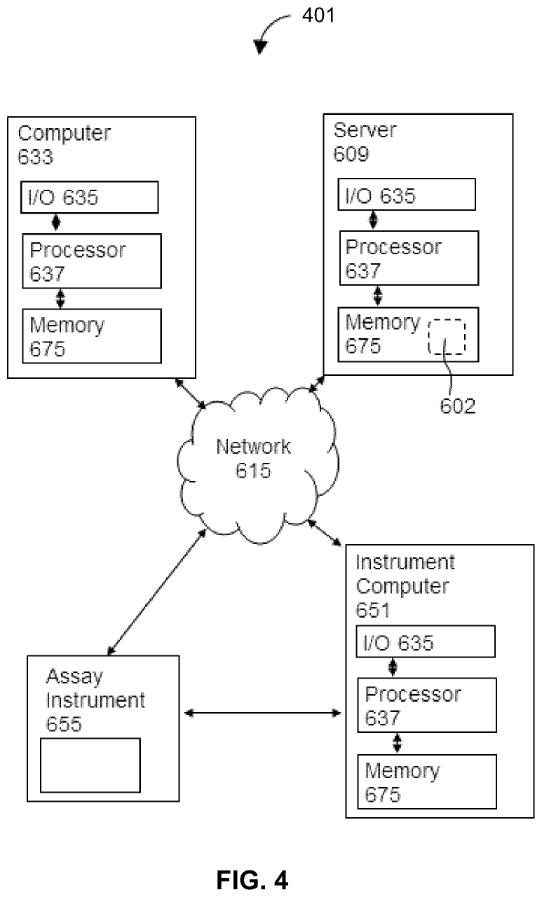

FIG. 4 shows an analysis system 401. The analysis system may include a machine learning subsystem 602 that has been trained on training data sets. In preferred embodiments, the machine learning subsystem performs the detecting 435. The system 401 includes at least one processor 637 coupled to a memory subsystem 675 including instructions executable by the processor 637 to cause the system 401 to detect 435 relevant signals; and to determine 439 a correlation to provide a predictive output.

The system 401 includes at least one computer 633. Optionally, the system 401 may further include one or more of a server computer 609 one or more assay instruments 655 (e.g., a microarray, nucleotide sequencer, an imager, etc.), which may be coupled to one or more instrument computers 651. Each computer in the system 401 includes a processor 637 coupled to a tangible, non-transitory memory 675 device and at least one input/output device 635. Thus, the system 401 includes at least one processor 637 coupled to a memory subsystem 675. The components (e.g., computer, server, instrument computers, and assay instruments) may be in communication over a network 615 that may be wired or wireless and wherein the components may be remotely located or located in close proximity to each other. Using those mechanical components, the system 201 is operable to receive or obtain training data such (e.g., images and molecular assay data) and outcome data as well as test sample data generated by one or more assay instruments or otherwise obtained. The system may use the memory to store the received data as well as the machine learning system data which may be trained and otherwise operated by the processor.

The memory subsystem 675 may contain one or any combination of memory devices. A memory device is a mechanical device that stores data or instructions in a machine-readable format. Memory may include one or more sets of instructions (e.g., software) which, when executed by one or more of the processors of the disclosed computers can accomplish some or all of the methods or functions described herein.

Using the described components, the system 401 is operable to produce a report and provide the report to a user via an input/output device. An input/output device is a mechanism or system for transferring data into or out of a computer. Exemplary input/output devices include a video display unit (e.g., a liquid crystal display (LCD) or a cathode ray tube (CRT)), a printer, an alphanumeric input device (e.g., a keyboard), a cursor control device (e.g., a mouse), a disk drive unit, a speaker, a touchscreen, an accelerometer, a microphone, a cellular radio frequency antenna, and a network interface device, which can be, for example, a network interface card (NIC), Wi-Fi card, or cellular modem. The machine learning subsystem 602 has preferably trained on training data that includes training images and known marker quantities.

Any of several suitable types of machine learning may be used for one or more steps of the disclosed methods. Suitable machine learning types may include neural networks, decision tree learning such as random forests, support vector machines (SVMs), association rule learning, inductive logic programming, regression analysis, clustering, Bayesian networks, reinforcement learning, metric learning, and genetic algorithms. One or more of the machine learning approaches (aka type or model) may be used to complete any or all of the method steps described herein.

For example, one model, such as a neural network, may be used to complete the training steps of autonomously identifying features and associating those features with certain outcomes. Once those features are learned, they may be applied to test samples by the same or different models or classifiers (e.g., a random forest, SVM, regression) for the correlating steps. In certain embodiments, features may be identified and associated with outcomes using one or more machine learning systems and the associations may then be refined using a different machine learning system. Accordingly some of the training steps may be unsupervised using unlabeled data while subsequent training steps (e.g., association refinement) may use supervised training techniques such as regression analysis using the features autonomously identified by the first machine learning system.

In decision tree learning, a model is built that predicts that value of a target variable based on several input variables. Decision trees can generally be divided into two types. In classification trees, target variables take a finite set of values, or classes, whereas in regression trees, the target variable can take continuous values, such as real numbers. Examples of decision tree learning include classification trees, regression trees, boosted trees, bootstrap aggregated trees, random forests, and rotation forests. In decision trees, decisions are made sequentially at a series of nodes, which correspond to input variables. Random forests include multiple decision trees to improve the accuracy of predictions. See Breiman, 2001, Random Forests, Machine Learning 45:5-32, incorporated herein by reference. In random forests, bootstrap aggregating or bagging is used to average predictions by multiple trees that are given different sets of training data. In addition, a random subset of features is selected at each split in the learning process, which reduces spurious correlations that can results from the presence of individual features that are strong predictors for the response variable. Random forests can also be used to determine dissimilarity measurements between unlabeled data by constructing a random forest predictor that distinguishes the observed data from synthetic data. Id.; Shi, T., Horvath, S. (2006), Unsupervised Learning with Random Forest Predictors, Journal of Computational and Graphical Statistics, 15(1):118-138, incorporated herein by reference. Random forests can accordingly by used for unsupervised machine learning methods of the invention.

In preferred embodiments, the machine learning subsystem 602 uses a neural network. Preferably, the machine learning subsystem 602 includes a deep-learning neural network that includes an input layer, an output layer, and a plurality of hidden layers.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for assessing cancer prognosis, the method comprising the steps of
identifying RNA expression levels present in a tissue or body fluid sample obtained from a cancer patient;
comparing said expression levels to expression levels expected in one or more stages of cancer;
correlating said identified expression levels with said expression levels expected in said one or more stages of cancer in order to generate an index score; and
assessing cancer prognosis based on the index score.

2. The method of claim 1, further comprising isolating an extracellular vesicle from the blood sample and identifying RNA expression levels for contents of the vesicle.

3. The method of claim 1, wherein identifying RNA expression levels comprises determining an amount of mRNA molecules by sequencing or microarray analysis.

4. The method of claim 3, wherein determining comprises interrogating the sample with probes specific for a panel of genes and measuring expression levels for positive probe responses.

5. The method of claim 4, wherein the panel of genes comprises genes previously identified as being overexpressed in cancer patients with a 5 year survival rate greater than 75%.

6. The method of claim 1, wherein identifying RNA expression levels involves making a whole transcriptome amplification library of mRNA molecules from the sample and then making cDNA copies of the mRNA.

7. The method of claim 1, wherein the index score indicates that the patient is at a low risk distant metastasis within 5 years.

8. The method of claim 1, further comprising analyzing a stained tissue sample from the patient to support or confirm the prognosis.

9. The method of claim 1, further comprising the step of providing the identified RNA expression levels as inputs to an analysis system trained on training data comprising one or more sets of training expression level measurements associated with known patient outcomes.

10. The method of claim 9, further comprising providing image data from the patient as part of the inputs to the analysis system, wherein the analysis system performs the comparing and correlating steps and then assesses cancer prognosis based on a combination of the image data and the comparing and correlating steps.

11. The method of claim 10, wherein the image data comprises an image of a stained tissue sample from the patient.

12. The method of claim 10, wherein the analysis system comprises a computer system with a machine learning algorithm.

13. The method of claim 1, wherein the comparing and correlating steps are performed exclusively on RNA expression levels substantially above a pre-determined threshold that is identified as background noise.

14. The method of claim 1, wherein the prognosis is that the patient is at a low risk of having metastatic cancer or recurrence of cancer.

15. The method of claim 1, wherein the index score is provided on a report to a physician and wherein the index score is on an easily interpretable scale due to the index score being a numerical value between −1.0 and 1.0.
